(19) United States
(12) Patent Application Publication
(10) Pub. No.: US 2007/0173540 A1
(43) Pub. Date: Jul. 26, 2007

(54) DNT-benzenesulfonate and methods of preparation thereof

(76) Inventors: Santiago Ini, Haifa (IL); Mili Abramov, Givataim (IL); Tamas Koltai, Netanya (IL)

Correspondence Address: KENYON & KENYON LLP, ONE BROADWAY, NEW YORK, NY, 10004, US

(21) Appl. No.: 11/525,335

(22) Filed: Sep. 21, 2006

Related U.S. Application Data
(60) Provisional application No. 60/761,562, filed on Jan. 23, 2006.

(51) Int. Cl. A61K 31/381; C07D 333/22

(52) U.S. Cl. 514/438; 549/76

(57) ABSTRACT

(S)—N,N-Dimethyl-3-(1-naphthalenyloxy)-3-(2-thienyl)propanamine benzenesulfonate (DNT-benzenesulfonate) and polymorphs of DNT-benzenesulfonate, compositions of DNT-benzenesulfonate and its polymorphs, processes for the preparation of DNT-benzenesulfonate and its polymorphs, and processes for the preparation of duloxetine hydrochloride from DNT-benzenesulfonate are provided.

### CROSS-REFERENCE TO RELATED APPLICATIONS

The present application claims the benefit of the following U.S. Provisional Patent Application No. 60/761,562 filed Jan. 23, 2006. The contents of which are incorporated herein by reference.

### FIELD OF THE INVENTION

The invention is directed to an intermediate for the synthesis of duloxetine. In particular, the invention is directed to the duloxetine intermediate DNT-benzenesulfonate, to the solid state chemistry of DNT-benzenesulfonate, and to processes for preparing DNT-benzenesulfonate and to converting DNT-benzenesulfonate into duloxetine HCl.

### BACKGROUND OF THE INVENTION

Duloxetine HCl (duloxetine hydrochloride) is a dual reuptake inhibitor of the neurotransmitters serotonin and norepinephrine. It is used for the treatment of stress urinary incontinence (SUI), depression, and pain management. Duloxetine hydrochloride is known by the chemical name (S)-(+)-N-methyl-3-(1-naphthalenyloxy)-3-(2-thienyl)propanamine hydrochloric acid salt, and has the following structure.

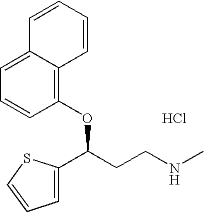

Duloxetine, as well as processes for its preparation, is disclosed in U.S. Pat. No. 5,023,269. EP Patent No. 457559 and U.S. Pat. Nos. 5,491,243 and 6,541,668 also provide synthetic routes for the preparation of duloxetine. U.S. Pat. No. 5,023,269 discloses preparing duloxetine by reacting (S)-(−)-N,N-Dimethyl-3-(2-thienyl)-3-hydroxypropanamine with fluoronaphtalene (Stage a), followed by demethylation with phenyl chloroformate or trichloroethyl chloroformate (Stage b) and basic hydrolysis (Stage c), according the following scheme.

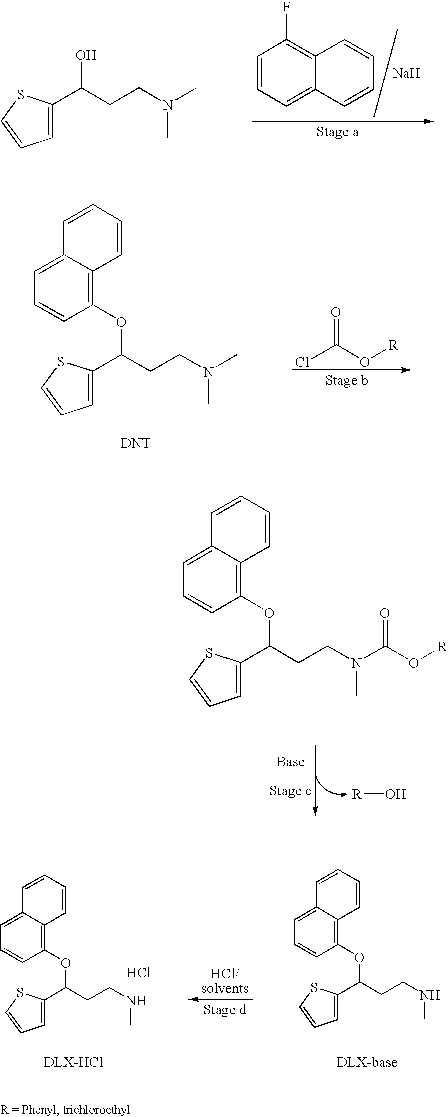

The conversion of duloxetine to its hydrochloride salt in ethyl acetate is described in U.S. Pat. No. 5,491,243 and in Wheeler, W. J., et al, J. Label. Cpds. Radiopharm, 1995, 36, 312.

As illustrated in the above scheme, DNT is an intermediate in the preparation of duloxetine. DNT has an N,N-dimethyl group instead of a secondary amine.

U.S. Pat. No. 5,023,269 describes the preparation of DNT-oxalate from DNT. See Example 1.

The oxalate salt of U.S. Pat. No. 5,023,269 is problematic for use on an industrial process. Oxalic acid has to be used to prepare the oxalate. Oxalic acid is highly toxic. Therefore, there is a need in the art to prepare duloxetine HCl with a process that is suitable for industrial scale.

### SUMMARY OF THE INVENTION

In one embodiment, the invention provides a compound (DNT-benzenesulfonate) having the following formula:

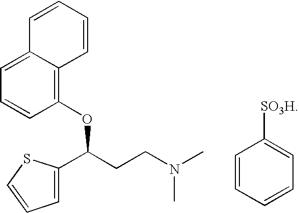

In another embodiment, the invention provides a process for preparing a pharmaceutically acceptable salt of duloxetine, comprising combining DNT, a solvent selected from the group consisting of C₁₋₈ alcohols, C₃₋₇ esters, C₃₋₈ ethers, C₃₋₇ ketones, C₆₋₁₂ aromatic hydrocarbons, acetonitrile, water and mixtures thereof with benzenesulfonic acid to form a reaction mixture, precipitating DNT-benzenesulfonate from the reaction mixture, converting the DNT-benzenesulfonate to DNT, converting the DNT to duloxetine, and converting the duloxetine to the pharmaceutically acceptable salt of duloxetine.

In another embodiment, the invention provides a crystalline form BSulfl of DNT-benzenesulfonate:

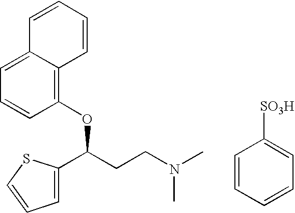

characterized by a powder XRD pattern with peaks at about 10.4°, 18.1°, 20.0°, 22.6°, and 23.1° 2θ±0.2° 2θ.

In another embodiment, the invention provides A process for preparing the crystalline form of claim 9 comprising combining benzenesulfonic acid with DNT in water to form a reaction mixture, precipitating the DNT-benzenesulfonate, and recovering the DNT-benzenesulfonate crystalline Form BSulfl.

### BRIEF DESCRIPTION OF THE FIGURE

FIG. 1 illustrates the powder X-ray diffraction pattern for DNT-benzenesulfonate Form BSulfl.

### DETAILED DESCRIPTION OF THE INVENTION

The present invention provides DNT-benzenesulfonate, which may be represented by the formula C₂₅H₂₇NO₄S₂ and the structure:

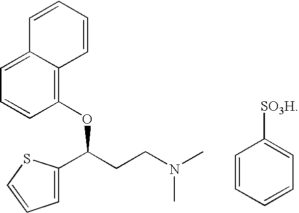

DNT-benzenesulfonate is preferably isolated as a solid, and, more preferably as a crystal.

DNT-benzenesulfonate can be characterized by data selected from: 1H NMR (400 MHz, DMSO d6) δ(ppm): 9.43 (s, 1H), 8.27 (dd, J1=6.16 Hz, J2=3.44 Hz, 1H), 7.84 (dd, J1=8.11 Hz, J2=3.17 Hz, 1H), 7.64 (m, 2H), 7.52 (m, 2H), 7.45 (d, J=7.04 Hz, 2H), 7.32 (m, 4H), 7.24 (d, J=3.28 Hz, 1H), 7.02 (d, J=7.72 Hz, 1H), 6.98 (t, J=4.68 Hz, 1H), 6.02 (t, J=6.28 Hz, 1H), 3.35 (m, 1H), 3.24 (m, 1H), 2.83 (s, 6H), 2.55 (m, 1H), 2.37 (m, 1H) 13C {1H}NMR (100 MHz): δ 152.4, 148.3, 143.7, 134.3, 128.8, 128.0, 127.7, 127.1, 126.7, 126.4, 126.2, 125.7, 121.9, 120.9, 107.8, 73.1, 54.1, 42.8, 33.2; and FAB MS: m/z 312 ([M−H]+, 100%).

The present invention also provides a process for preparing DNT-benzenesulfonate. DNT benzenesulfonate may be prepared by combining DNT and benzenesulfonic acid to create a reaction mixture. DNT-benzenesulfonate forms in such reaction mixture through contact of DNT with benzenesulfonic acid.

In one embodiment, a solution or suspension of DNT in a solvent is combined with benzenesulfonic acid to form a reaction mixture, followed by recovery of the DNT-benzenesulfonate from the mixture. DNT-benzenesulfonate may be prepared by dissolving DNT in a solvent, combining the resulting solution with benzenesulfonic acid to form a reaction mixture, and precipitating the DNT-benzenesulfonate from the mixture. The organic solvent may be selected from the group consisting of C₁₋₈ alcohols, C₃₋₇ esters, C₃₋₈ ethers, C₆₋₁₂ aromatic hydrocarbons, acetonitrile, water and mixtures thereof. Preferably, the solvent is water.

In one embodiment, DNT, benzenesulfonic acid and at least one solvent are combined to form a reaction mixture at about room temperature. DNT-benzenesulfonate then precipitates out of such mixture. Such precipitation may occur on its own or be induced. The reaction mixture may be stirred before, during or after precipitation.

The resulting precipitate from any of the above embodiments may be recovered by conventional techniques, such as filtration. The precipitate may be dried under ambient or reduced pressure, or elevated temperatures. In one embodiment, the precipitate is dried at room temperature, under vacuum conditions. In one embodiment, the precipitate is dried at 50° C., at a pressure less than about 100 mmHg.

The DNT-benzenesulfonate of the invention can be prepared in different polymorphic forms. Polymorphism, the occurrence of different crystal forms, is a property of some molecules and molecular complexes. A single molecule, such as DNT-benzenesulfonate may give rise to a variety of crystalline forms having distinct crystal structures and physical properties like melting point, X-ray diffraction pattern, infrared absorption fingerprint, and solid state NMR spectrum. One crystalline form may give rise to thermal behavior different from that of another crystalline form. Thermal behavior can be measured in the laboratory by such techniques as capillary melting point, thermogravimetric analysis (“TGA”), and differential scanning calorimetry (“DSC”), which have been used to distinguish polymorphic forms.

The difference in the physical properties of different crystalline forms results from the orientation and intermolecular interactions of adjacent molecules or complexes in the bulk solid. Accordingly, polymorphs are distinct solids sharing the same molecular formula, yet having distinct physical properties that can be advantageous in certain applications compared to other crystalline forms of the same compound or complex. Therefore, processes for the preparation of polymorphic forms of DNT-benzenesulfonate are desirable.

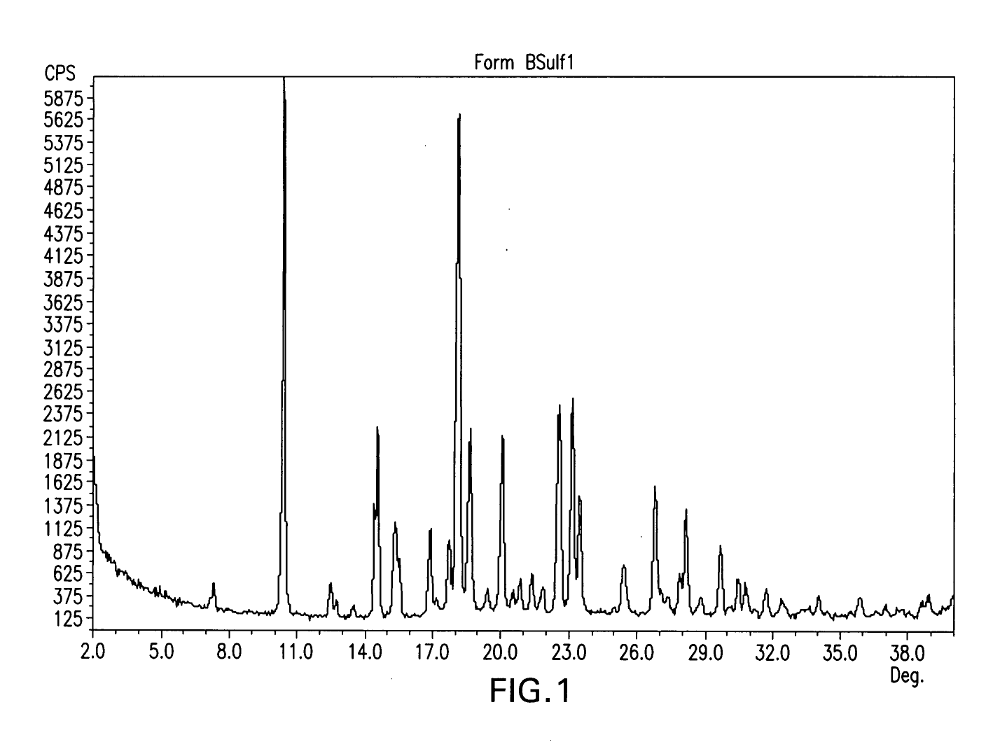

One such crystalline form of DNT-benzenesulfonate, herein defined as Form BSulfl, is characterized by a powder XRD pattern with peaks at about 10.4°, 18.1°, 20.0°, 22.6°, and 23.1° 2θ±0.2° 2θ. The crystalline form may be further characterized by X-ray powder diffraction peaks at about 14.5°, 18.7°, 23.5°, 26.8°, and 28.1° 2θ±0.2° 2θ. DNT-benzenesulfonate Form BSulfl can also be characterized by an X-ray powder diffraction pattern, substantially as depicted in FIG. 1.

Form BSulfl may be prepared according to the processes set out above.

Preferably, the DNT-benzenesulfonate, Form BSulfl resulting from the processes described above is present in a composition, such as a batch, having a polymorphic purity of at least about 10 percent by weight, more preferably, at least about 25 percent by weight, and most preferably at least about 50 percent by weight of a single crystalline form.

The DNT-benzenesulfonate of the invention, including Form BSulfl, will generally have a maximal particle size of less than about 500 μm, preferably less than about 300 μm, more preferably less than about 200 μm, and most preferably less than about 100 μm. It is particularly preferred that crystalline DNT-benzenesulfonate have a maximal particle size of less than about 50 μm. The particle size of DNT-benzenesulfonate crystalline forms may be measured by methods including, but not limited to sieves, sedimentation, electrozone sensing (coulter counter), microscopy, and Low Angle Laser Light Scattering (LALLS).

The DNT-benzenesulfonate of the present invention is useful as an intermediate in the preparation of pharmaceutically acceptable salts of duloxetine, particularly the hydrochloride salt. The conversion can be carried out by combining DNT-benzenesulfonate, water, a base such as ammonium hydroxide, and toluene to obtain a two phase system, separating the organic phase containing DNT and toluene, and converting the DNT to duloxetine HCl. The DNT-benzenesulfonate used in this process is preferably the DNT-benzenesulfonate prepared as described above.

The conversion of DNT to a pharmaceutically acceptable salt of duloxetine may be performed by any method known in the art, such as the one described in U.S. Pat. No. 5,023,269 or in co-pending U.S. patent application Ser. No. 11/318,365, filed on Dec. 23, 2005, for making duloxetine HCl. Preferably, the conversion is performed by dissolving DNT in an organic solvent, and combining it with an alkyl haloformate. That step will yield duloxetine alkyl carbamate, which can be combined with an organic solvent and a base, to yield duloxetine. The duloxetine may then be converted to a pharmaceutically acceptable salt. More preferably, the conversion is performed by dissolving DNT in a water immiscible organic solvent; adding alkyl chloroformate at a temperature of about 5° C. to less than about 80° C. to obtain duloxetine alkyl carbamate, combining the duloxetine alkyl carbamate with an organic solvent and a base; maintaining the reaction mixture at reflux temperatures for at least 1 to 3 hours; cooling, and adding water and an additional amount of an organic solvent; recovering duloxetine; combining the duloxetine with a solvent; adding hydrochloric acid until a pH of about 3 to about 4 is obtained; maintaining the reaction mixture to obtain a solid residue; and recovering duloxetine HCl.

The following non-limiting examples are merely illustrative of the preferred embodiments of the present invention, and are not to be construed as limiting the invention, the scope of which is defined by the appended claims.

### Instruments

X-Ray powder diffraction (XRD) data was obtained using a Scintag X-ray powder diffractometer model X'TRA equipped with a Cu-tube solid state detector. A round standard aluminum sample holder with rough zero background quartz plate with a cavity of 25 (diameter)×0.5 mm (depth) was used. The scanning parameters included: range: 2° to 40° 2θ; scan mode: continuous scan; step size: 0.05°; and a rate of 5°/minute.

### EXAMPLES

### Preparation of DNT-benzenesulfonate

### Example 1

Benzenesulfonic acid (2.4 g) was added to 4 g of DNT in 30 ml of water, and the mixture was stirred for an additional 1 hour, filtrated, and washed with water. After drying in a vacuum oven (10 mm Hg) at 50° C. for 16 hours, 1.5 g (67.5% yield), of product were obtained. The product was analyzed by XRD, and found to be Form BSulfl after the drying.

### Preparation of DNT

### Example 2

A 2 liter reactor equipped with mechanical stirrer is charged with a mixture of 107 g DNT-benzenesulfonate, 600 ml water, 96 ml of a 22 percent solution of ammonium hydroxide, and 1 liter of toluene. The mixture is stirred at 25° C. for 20 to 30 minutes, and the organic phase is separated and washed with water (3×300 ml). The toluene solution of DNT can be used to form duloxetine hydrochloride without evaporation.

### Example 3

A 100 ml three necked flask, equipped with mechanical stirrer, thermometer, dean stark, and condenser, was charged with 5 g of DNT and 25 ml of toluene. The clear solution was heated, and an azeotropic distillation was performed for about 30 to about 60 minutes. After cooling to room temperature, 4.6 ml of ethyl chloroformate were added during over a period of 1 to 2 hours, and the reaction mixture was stirred at room temperature over night.

Diluted NH₄OH was added to the reaction mixture, which was stirred for an additional 30 minutes. After phase separation, the organic phase was washed with water (3×20 ml), dried over Na₂SO₄, filtered, and concentrated to dryness to give 5.2 g of a brownish oil. (88% chemical yield).

### Example 4

A 100 ml three necked flask equipped, with mechanical stirrer, thermometer, and condenser, was charged with 2.5 g duloxetine ethyl carbamate and 20 ml toluene. The mixture was stirred, and 4.8 g of KOH were added in portions, followed by reflux for about 3 hours.

After cooling, 30 ml of water, followed by 20 ml of toluene, were added, and the resulting organic phase was washed with water (3×20 ml), dried over Na₂SO₄, filtered and concentrated to dryness to give 1.70 g of an oily product. (85.31% yield).

### Example 5

To a solution of 1 g of duloxetine in 10 ml MEK was slowly added 0.32 ml of a 37 percent hydrochloric acid solution. The mixture was stirred until a solid formed. The resulting solid was filtered, and dried in a vacuum oven to give 0.50 g of (S)-(+)-duloxetine hydrochloride. (94.64% yield).

While it is apparent that the invention disclosed herein is well calculated to fulfill the objects stated above, it will be appreciated that numerous modifications and embodiments may be devised by those skilled in the art. Therefore, it is intended that the appended claims cover all such modifications and embodiments as falling within the true spirit and scope of the present invention.

What is claimed:

1. A compound (DNT-benzenesulfonate) having the following formula:

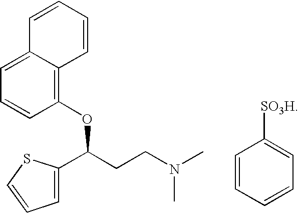

2. The compound of claim 1, wherein the compound is isolated.

3. The compound of claim 2, wherein the compound is isolated as a crystal.

4. A process for preparing DNT-benzenesulfonate of claim 1, comprising combining DNT with benzenesulfonic acid to form a reaction mixture, precipitating DNT-benzenesulfonate from the reaction mixture and recovering the precipitate.

5. The process of claim 4 wherein the reaction mixture contains a solvent selected from the group consisting of C₁₋₈ alcohols, C₃₋₇ esters, C₃₋₈ ethers, C₆₋₁₂ aromatic hydrocarbons, acetonitrile, water, and mixtures thereof.

6. The process of claim 5, wherein the solvent is water.

7. A process for preparing a pharmaceutically acceptable salt of duloxetine, comprising combining DNT, a solvent selected from the group consisting of C₁₋₈ alcohols, C₃₋₇ esters, C₃₋₈ ethers, C₃₋₇ ketones, C₆₋₁₂ aromatic hydrocarbons, acetonitrile, water and mixtures thereof with benzenesulfonic acid to form a reaction mixture, precipitating DNT-benzenesulfonate from the reaction mixture, converting the DNT-benzenesulfonate to DNT, converting the DNT to duloxetine, and converting the duloxetine to the pharmaceutically acceptable salt of duloxetine.

8. The process of claim 7, wherein the pharmaceutically acceptable salt of duloxetine is duloxetine HCl.

9. A crystalline form of DNT-benzenesulfonate: 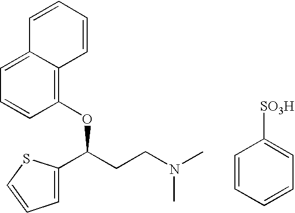
characterized by a powder XRD pattern with peaks at about 10.4°, 18.1°, 20.0°, 22.6°, and 23.1° 2θ+0.2° 2θ.

10. The crystalline form of claim 9, further characterized by X-ray powder diffraction peaks at about 14.5°, 18.7°, 23.5°, 26.8°, and 28.1° 2θ±0.2° 2θ.

11. The crystalline form of claim 9 further characterized by an X-ray powder diffraction pattern substantially as depicted in FIG. 1.

12. The crystalline form of claim 9, wherein the crystalline form is present in a batch at a polymorphic purity level of at least about 50% by weight.

13. A process for preparing the crystalline form of claim 9 comprising combining benzenesulfonic acid with DNT in water to form a reaction mixture, precipitating the DNT-benzenesulfonate, and recovering the DNT-benzenesulfonate crystalline Form BSulfl.

14. The process of claim 13, wherein the process is carried out at about room temperature.

15. A process for preparing duloxetine hydrochloride comprising combining benzenesulfonic acid with DNT in water to form a reaction mixture, precipitating the DNT-benzenesulfonate, and converting the crystalline DNT-benzenesulfonate to duloxetine hydrochloride.

16. A process for preparing a pharmaceutically acceptable salt of duloxetine, comprising converting DNT-benzenesulfonate prepared by the process of claim 1 to the pharmaceutically acceptable salt of duloxetine.

17. The process of claim 16, wherein the pharmaceutically acceptable salt of duloxetine is duloxetine hydrochloride.
